(19) United States
(12) Patent Application Publication
(10) Pub. No.: US 2007/0092961 A1
(43) Pub. Date: Apr. 26, 2007

(54) Process to cultivate Brevundimonas diminuta for filtration validation

(76) Inventor: Diego Vargas, Lansdale, PA (US)

Correspondence Address: MERCK AND CO., INC, P O BOX 2000, RAHWAY, NJ, 07065-0907, US

(21) Appl. No.: 11/544,350

(22) Filed: Oct. 6, 2006

Related U.S. Application Data
(60) Provisional application No. 60/724,350, filed on Oct. 6, 2005.

(51) Int. Cl. C12N 1/20

(52) U.S. Cl. 435/252.3

(57) ABSTRACT

The present invention relates to a method for culturing Brevundimonas diminuta for filtration validation. The method comprises inoculating the B. diminuta cells in an appropriate medium, growing the inoculated medium in a gas-impermeable chamber, wherein there is room for air in the headspace of the chamber, wherein air is continually passed through the headspace, and rocking the chamber to induce wave in the medium.

### CROSS-REFERENCE TO RELATED APPLICATIONS

### Background of the Invention

Sterile products need to be manufactured in sterile processes. Filtration is an effective method for sterilization, especially for heat liable pharmaceuticals and biologicals. The validation of the filtration is required by United States Food and Drug Administration (FDA). To fulfill the requirements of sterile filtration, a filter must be able to remove from the filtration stream at least 1×10⁷ CFU/cm² of the challenge organism, Brevundimonas diminuta, and produce a sterile effluent. (Fennington, et al., PDA Journal of pharmaceutical Science & Technology, 51:153-155 (1997))

Brevundimonas diminuta (ATTC#19146), formerly known as Pseudomonas diminuta, is an aerobic gram-negative bacteria. Because of its small size, B. diminuta is a standard microbial organism for validation of membrane filters for sterilization.

The B. diminuta cells ideal for performing filter validation should have high cell concentration, very small cell size and a mono-dispersed population. B. diminuta cells are usually cultivated with deep fermentation techniques according to ASTM F838-83 procedure (ASTM Designation: F838-83 Standard Test Method for Determining Bacterial Retention of Membrane Filters Utilized for Liquid Filtration, p 938-944). The final batch is grown aerobically to early stationary phase (approximately 2×10¹⁰ CFU/mL). However, this deep fermentation procedure often leads to aggregated and larger cells. Thus, there is a need for a better method for the production of B. diminuta cells suitable for the validation of sterilizing grade filter membranes.

The references cited herein are not admitted to be prior art to the claimed invention.

### SUMMARY OF THE INVENTION

The present invention relates to a method of culturing Brevundimonas diminuta for filtration validation comprising. The method comprises inoculating the B. diminuta cells in an appropriate medium, growing the inoculated medium in a gas-impermeable chamber, wherein there is room for air in the headspace of the chamber, wherein air is continually passed through the headspace, and rocking the chamber to induce wave in the medium. According to a preferred embodiment, the method further comprises harvesting the B. diminuta cells with a tangential filtration cassette system.

According to an embodiment of the present invention, the medium is a minimum essential medium with a high osmolarity. The medium is preferably saline-lactose broth. The pH of the medium can be controlled with the content of carbon dioxide in the passed-through air.

According to a preferred embodiment of the present invention, the headspace is about one half of the volume of the chamber. The chamber is preferably a disposable pre-sterilized bag.

According to an embodiment of the present invention, the chamber comprises vent filters, an inlet port, a pressure regulator, a sample port, and an oxygen port.

The chamber is preferably rocked at a rate of 15 rocking/minute.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1. Growth curve of B. diminuta in filter sterilized SLB: Rocking versus Static culture.

FIG. 2. Cultivation of B. diminuta in autoclaved growth medium A and saline lactose broth with static method.

### DETAILED DESCRIPTION OF THE INVENTION

The present invention relates to a method to produce cell paste of Brevundimonas diminuta suitable for the validation of membrane filters for sterilization. The B. diminuta cells are preferably produced in a disposable bioreactor.

### 1. The B. diminuta Cells

The B. diminuta cells for the filtration validation are in a high concentration, and have very small size and high mono-dispersion. Preferably, the B. diminuta cells are B. diminuta, ATTC#19146.

The cell size of B. diminuta is critical for the determination of retention characteristics of the membrane filters to be validated. The B. diminuta cell paste produced with the present invention has a size specification of 0.4-1.0 μm in diameter. More preferably, the cell size of B. diminuta is about 0.6×0.4 μm² . The cell paste of B. diminuta is commercially available. (e.g., the cell paste from Alberta Research Council) Nevertheless, the cell sizes of such cell paste obtained from deep fermentation culturing techniques are often out of the range of 0.4-1.0 μm in diameter.

The cell size of B. diminuta is influenced by many factors of growth conditions, including medium, agitation rate, (Lee, et al, PDA Journal of Pharmaceutical Science & Technology, 56:99-108 (2002)), and aeration. The cell size of B. diminuta can be determined with different approaches, such as micro-filtration. According to an embodiment of the present invention, the B. diminuta cells are capable of being retained by the 0.2μ filter, but not completely retained by the 0.45μ filter.

To be used for filtration validation, the B. diminuta cells also need to be in a high concentration (preferably >1×10⁸ CFU/mL), and have high mono-dispersion (preferably >80%), high viability (preferably >90%), and high bacteriological purity. These characteristics can be determined using the methods including direct microscopic count, standard plate count, gram stain, streak plate, scanning electron microscopy, and biochemical identification. (Fennington, et al., PDA Journal of Pharmaceutical Science & Technology, 51:153-155 (1997))

### 2. Medium

The B. diminuta cells can be reconstituted and checked for purity by the streak plate method on Tryptic Soy Agar plates (Remel Microbiology Products, Lenexa, Kans.) at 32° C. The cells can then cultured in the medium such as medium A and saline-lactose broth.

The B. diminuta cells of the present invention are preferably grown in a minimum essential medium with a high osmolarity to control the cell size and the dispersion characteristics.

Traditionally, microorganisms, such as B. diminuta, are cultivated using growth medium A (7.5 g of Tryticase Peptone, 2.5 g of Yeast Extract, 0.5 g of Sodium Chloride and 0.35 g of magnesium sulfate added to 1.0 L of hot distilled water) according to ASTM F838-83 procedure (ASTM Designation: F838-83 Standard Test Method for Determining Bacterial Retention of Membrane Filters Utilized for Liquid Filtration, p 938-944).

The preparation of B. diminuta cells preferably employs saline-lactose broth (1.3 g of Lactose Broth dry powder in 100 mL of hot distilled water with 970 mL of sodium chloride solution). B. diminuta cells grown in saline-lactose broth have a small size due to osmotic pressure constraints. On the other hand, it is often difficult to cultivate B. diminuta cells in high titers in saline-lactose broth, because this medium is low in nutrients. In contrast, B. diminuta cells grown in medium A have a high titers but a larger size, because medium A is more nutrient rich than saline-lactose broth.

Cell paste medium requires the use of harvest buffer, composed of potassium phosphate monobasic, potassium phosphate dibasic and glycerol solution.

### 3. The Bioreactor

The fermenter used in the present invention is preferably able to be operated and kept enclosed in an incubator or benchtop. According to an embodiment of the present invention, the fermenter is the Wave Bioreactor®. (Wave Biotech LLC, Bridgewater, N.J., http://www.wavebiotech.com/)).

The Wave Bioreactor® comprises a fermentation chamber and a rocking platform. The fermentation chamber of Wave Bioreactor® is a disposable pre-sterilized bag, such as CellMate®, or Cellbag®, which is placed on the special rocking platform. Culture medium and cells are contained in the Cellbag®. The Cellbag® is equipped with vent filters, inlet port, a pressure regulator, sample port, and OxyProbe® port. Those equipments allow the inlet of air to keep the inflated bag supported on the rocking platform, maintains the inflated bag at a low pressure, and supply oxygen to the culture medium.

The Wave Bioreactor® is an ideal device for cell culture. When operating, the rocking motion of Wave Bioreactor® platform induces waves in the culture fluid inside the Cellbag®. These waves promote mixing and transfer of oxygen to the culture fluid, resulting in a perfect environment for cell growth. While widely used in the cultivation of mammalian cells, the Wave Bioreactor® can also be used in the cultivation of microbial cells, such as yeast and anaerobic organisms. According to a preferred embodiment of the present invention, the Wave Bioreactor® is used for the cultivation of Brevundimonas diminuta cells for filtration validation.

The Wave Bioreactor® can be used to solve the problem of scaling up the growth of relatively large quantities of B. diminuta cells. The reactor does not occupy a large space and it could be fully instrumented for monitoring cell growth parameters. The Cellbag® is only filled with fifty percent of its total volume and the rocking motion of the platform can provide the mixing required to grow and aerate the organism, which can easily reach the concentration of over 20×10⁶ cells/ml. Moreover, the bioreactor requires no cleaning or sterilization, providing the ease in operation and protection against cross-contamination.

For instance, ten liter of appropriate medium can be added to a 20-L Wave Bioreactor®. The reactor moves in a rocking motion at a speed of 15 rocking/minute while aeration rate is maintained at 0.8 L/min for 28 hours.

### 4. Cell Harvesting

According to the ASTM Standard outlines, continuous centrifugation is used for harvesting B. diminuta, with a yield of about 30%. Preferably, B. diminuta cells are harvested using tangential filtration cassette system, which leads to an improvement of yield to 90% yield. The Centramate™ (Pall Filtron, Inc) can be used to collect the B. diminuta cells to obtain the cell paste.

### 5. Embodiments

The present invention can be used to prepare frozen Brevundimonas diminuta cell paste applicable in filter validation studies. The technology can also be used for any microbial cell growth where the cell size and the mono-dispersion are critical process variables that need to be controlled.

Many constraints have limited the scale-up capability for on-site cultivation of B. diminuta, which is often surrounded by delicate tissue culture of certain vaccines operations. Thus, the use of a 150 L standard fermentation is prohibited from the safety perspective and from the additional cost involved in the purchase of the chamber, instrumentation and utility supply. The present invention solved this problem, and can be used to obtain approximately 100 liters of fermentation broth.

According to an embodiment of the present invention, the B. diminuta cells are aerated on the liquid surface with very gentle agitation using bioreactor with a disposable fermentation chamber. The medium such as saline lactose broth together with the gentle agitation induces cell growth to a high concentration (>1×10⁸ CFU/mL), however, with very small size (0.6×0.4 μm) and higher mono-dispersion (>80%). The optimal harvest time for the organism decreased from 36 hours to 28 hours. The recovery is improved to 78% using tangential filtration directly connected to the bag versus 20% yield obtained via centrifugation.

The product can be in the form of a frozen cell paste that can be used, after reconstituting in an appropriate buffer solution, to provide Bio-sterile Validation the ability to perform Microbial Retention Test with filter cartridges and other filter configurations without cultivating large quantities of inoculum. According to an embodiment of the present invention, the total cell viability time can be extended to 120 days frozen at −70° C. The cells do not lose their viability over this period of time, being ready to use whenever needed.

### EXAMPLES

Examples are provided below to further illustrate different features of the present invention. The examples also illustrate useful methodology for practicing the invention. These examples do not limit the claimed invention.

Experimental Procedure:

Saline lactose broth was made as follows: 1.3 g of Lactose Broth dry powder in 100 mL of hot distilled water with 970 mL of sodium chloride solution.

Growth medium A was made according to ASTM method: dissolve in WFI and dilute to 1.0 L Tyrpticase Peptone (7.5 g), Yeast Extract (2.5 g), Sodium Chloride (0.5 g), and Magnesium Sulfate (0.35 g).

Harvesting Buffer: Dissolve in 100-mL of glycerol Mono-basic Potassium Phosphate (0.79 g) and K₂HPO₄ (1.0 g). Adjust pH to 7.2 with 0.1 N KOH. Dilute to 1.0 L with WFI.

The Media were either autoclaved at 121° C. for 15 minutes, or filter-sterilized using a Millipak 40.

### Example 1

### The Growth of B. diminuta Cells

ATCC freeze-dried Brevundimonas diminuta cells (ATCC #19146) were reconstituted and checked for purity by the streak plate method on Tryptic Soy Agar plates (Remel Microbiology Products, Lenexa, Kans.) at 32° C. Once B. diminuta was transferred from the Tryptic Soy Agar plates to Soybean Casein Digest (CM 490), the work on the Wave Bioreactor® was initiated.

Both autoclaved and filter-sterilized saline lactose broth were used in the experiment respectively. For each, the medium was aseptically loaded into the CellMate™ bag using a peristaltic pump into a 2.0 L CellMate™ bag with a working volume of 1.0 L. The working seed of B. diminuta in Soybean Casein Digest Broth was inoculated to the saline lactose broth in a ratio of 4 mL/L, using a syringe through the inlet port.

Two modes of incubation were chosen, static and rocking mode for both autoclaved and filter-sterilized media. The conditions for the rocking mode were chosen based on the manufacturing recommendations supplied with the Wave™ Bioreactor (WaveBiotech™) of 0.8 L/min for oxygen flow rate and a speed of 15 rocking per minute. The temperature of incubation was maintained at 30±2° C., and the time of incubation was 40 hours.

During the incubation, samples were taken at timed intervals through the sampling port for enumeration and cell size determination. Each sample was removed through the sample port using a syringe according to the manufacturer's instructions. A growth curve was established from the timed enumerations.

Alternatively, exactly the same procedure was followed with the exception of the method of sterilization of saline-lactose broth. A 1.3 L of the saline-lactose broth was prepared using a Millipak 40 as the method of sterilization. A filter flush of 300 mL was discarded before the tubing is connected to the inlet of the bag.

B. diminuta cells were also grown in growth medium A, following the same procedure as that of saline lactose broth.

### Example 2

### The Analysis of the B. diminuta Cell Growth

When grown in saline lactose broth, B. diminuta cells have a lag period of approximately 10 hours before the exponential phase is achieved. The enumerations obtained at the early stationary phase with filter-sterilized and autoclaved Saline Lactose Broth were similar. It was also observed that the enumeration began to decline after about 40 hours of incubation. The optimal harvest time for the organism was determined to be 28±2 hours for both methods of sterilization.

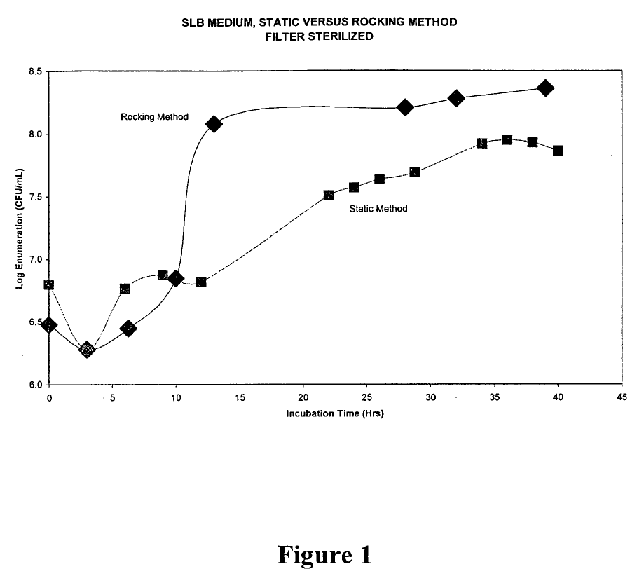

For the growth in filter-sterilized Saline Lactose Broth, the static and rocking mode of the cultivation was compared (FIG. 1). Less samples were taken for enumeration in the rocking method than that of the static method. The growth phase lasted the same time, however the rocking method increased the cell concentration to a minimum of 1.0 log versus that of the static mode.

The average cell size remained constant during both static and rocking mode as it is shown in Table 1. The size of B. diminuta cells was determined using ocular micrometer. Mondispersion was determined by optical microscopy.

TABLE 1
Summary of Average Sizing and Monodispersion of B. diminuta Grown
Using the Experimental Matrix in the Cell Paste Project Plan

| | Sterilization | | Average | |
|---|---|---|---|---|
| | Method | | Monodispersion | Average Sizing |
| Media Used | Of Media | Incubation Method | Over 40 Hours* | Over 40 Hours* |
| saline lactose broth | Filtered | Static | 95% | 0.7 μm × 0.4 μm |
| saline lactose broth | Autoclaved | Static | 94% | 0.7 μm × 0.4 μm |
| saline lactose broth | Filtered | Rocking | 97% | 0.6 μm × 0.4 μm |
| saline lactose broth | Autoclaved | Rocking | 98% | 0.6 μm × 0.4 μm |
| growth medium A | Filtered | Static | 95% | 1.5 μm × 0.5 μm |
| growth medium A | Autoclaved | Static | 96% | 1.6 μm × 0.5 μm |

*Average monodispersion and average sizing calculated using data obtained during entire incubation.

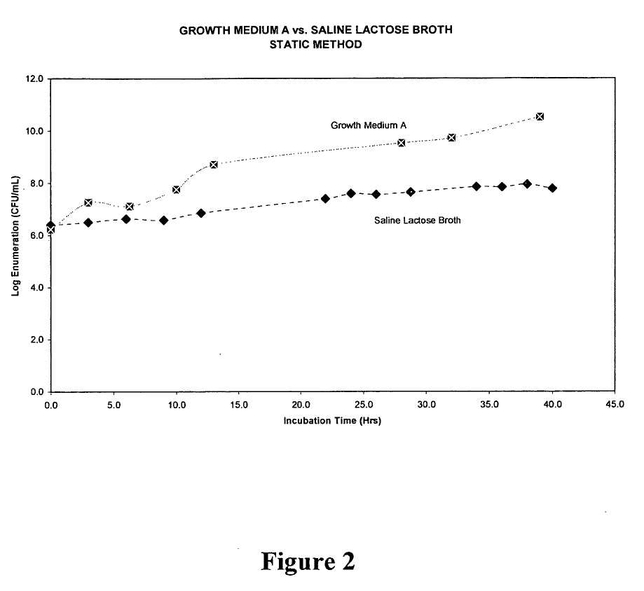

A comparison of the medium of cultivation followed the experimental matrix to determine which medium would increase the enumeration without sacrificing the cell size. FIG. 2 shows the results.

Growth medium A showed a greater capacity to increase the enumeration of the cell versus saline lactose broth. Growth medium A is composed of Trypticase® Peptone and Yeast Extract, therefore a higher carbon source is translated into a more efficient utilization of the source to translate it into cell division, versus saline lactose broth which it is classified as a minimal nutrient medium. However, the cell size of B. diminuta grown in growth medium A was almost double in length, and not within the current specifications (as shown in Table 1).

### Example 3

### Large-Scale Growth of B. diminuta Cells and Cell Harvesting

Based on the previous results, saline lactose broth was selected as the optimum medium on a rocking mode to scale-up the cultivation of the organism. A 20 L CellMate™ disposable bag was used with 10 L of saline lactose broth. Three lots of cell paste were cultivated to harvest sufficient cell paste for the stability study and for future Microbial Retention experiments. The organism enumeration and cell size was very similar for the three consistency lots and very similar to the values obtained from the small-scale experiment.

Once the stationary phase was reached, the batches of B. diminuta cells were harvested using a Pall's Centramate™ tangential filtration cassette with an Omegas membrane.

The inlet port of the bag reactor was used as the outlet of the B. diminuta cells, and connected to the inlet port of the reservoir in the Centramate™. The B. diminuta cells were then be transferred from the bioreactor to the reservoir in the Centramate™. The CellMate™ bag was placed inside a Biological Safety Cabinet and connected to the reservoir of the Centramate™. Initially, 200 mL of saline lactose broth were in the reservoir with a retentate circulating flow rate of 800 mL/min (LMH).

Once the permeate valve was slightly opened, the inoculated saline lactose broth started to flow into the reservoir. The permeate flow rate was maintained at 90 mL/min (LMH). The transmembrane pressure was 4.0 psig during the concentration step. At the end of concentration step, 300 mL of saline lactose broth remained in the reservoir. The diafiltration step took place by adding cell paste harvesting buffer consisting of 100 mL of Glycerol, 0.79 g of mono-basic Potassium Phosphate, 1.0 g of dibasic Potassium Phosphate diluted to 1.0 L and pH adjusted to 7.2 with 0.1 N Sodium Hydroxide. Two 300 mL aliquots of harvest buffer were added to the reservoir to displace the saline lactose broth while keeping the highest circulation velocity in the retentate. A sample was taken from the reservoir for enumeration before adding and diluting the cell paste to a total volume of 600 mL. The recovery averaged 78% for the concentration step.

The cell paste was dispensed under the Biological Safety Cabinet into 25 mL aliquots and frozen at −70° C. Stability studies were conducted on each of the three lots of reconstituted cell paste produced by analyzing size, mono-dispersion and enumeration at after 24 hours, 55 days, 90 days and 120 days. Vials were drawn to reconstitute with Sodium Chloride solution.

Each lot of cell paste produced was found to be stable up to 120 days held at −70° C. All lots of cell paste once thawed and reconstituted 1:10 with Sodium Chloride have a concentration of approximately 1×10⁸ CFU/mL and meet the requirements for sizing and monodispersion.

Other embodiments are within the following claims. While several embodiments have been shown and described, various modifications may be made without departing from the spirit and scope of the present invention.

What is claimed is:

1. A method for culturing Brevundimonas diminuta, for filtration validation comprising,
inoculating the B. diminuta cells in an appropriate medium,
growing the inoculated medium in a gas-impermeable chamber, wherein there is room for air in the headspace of the chamber, wherein air is continually passed through the headspace, and
rocking the chamber to induce wave in the medium.

2. The method of claim 1 wherein the medium is a minimum essential medium with a high osmolarity.

3. The method of claim 2 wherein the medium is saline-lactose broth.

4. The method of claim 1 wherein the pH of the medium is controlled with the content of carbon dioxide in the passed-through air.

5. The method of claim 1 wherein the headspace is about one half of the volume of the chamber.

6. The method of claim 1 wherein the chamber is a disposable pre-sterilized bag.

7. The method of claim 1 wherein the chamber comprises vent filters, an inlet port, a pressure regulator, a sample port, and an oxygen port.

8. The method of claim 1 wherein the chamber is rocked at a rate of 15 rocking/minute.

9. The method of claim 1 further comprising harvesting the B. diminuta cells with a tangential filtration cassette system.
